(19) United States
(12) Patent Application Publication
(10) Pub. No.: US 2004/0009563 A1
(43) Pub. Date: Jan. 15, 2004

(54) Method of producing vertebrate host mimic with modified lipid based media

(76) Inventors: Teunis Dekker, Staffanstorp (SE); Agenor Mafra-Neto, Riverside, CA (US)

Correspondence Address: Eric L. Tanezaki, STETINA BRUNDA GARRED & BRUCKER, Suite 250, 75 Enterprise, Aliso Viejo, CA, 92656, US

(21) Appl. No.: 10/615,098

(22) Filed: Jul. 8, 2003

Related U.S. Application Data
(60) Provisional application No. 60/394,399, filed on Jul. 8, 2002.

(51) Int. Cl. C12P 7/64

(52) U.S. Cl. 435/134; 424/405; 424/84

(57) ABSTRACT

A vertebrate host mimic is provided wherein the same is produced according to the method comprising the steps of (1) providing lipid based media, (2) providing microorganisms, the microorganisms being associated with the skin of the host vertebrate and operative to excrete sub-products which modify the media upon combination with the lipid based media to produce modified lipid based media, (3) combining the lipid based media and the microorganisms to produce lipid based media, and (4) collecting the modified lipid based media.

### CROSS-REFERENCE TO RELATED APPLICATIONS

This application claims the benefit of U.S. Provisional Application No. 60/394,399, filed Jul. 8, 2002. Additionally, U.S. Provisional Application No. 60/394,399 is expressly incorporated herein by reference.

### STATEMENT RE: FEDERALLY SPONSORED RESEARCH/DEVELOPMENT

Not Applicable

### BACKGROUND OF THE INVENTION

The present invention relates generally to substances which modify a behavior of arthropods which are parasitic to a vertebrate host, and more particularly, to a lipid based media which when modified produces an odor to modify the behavior of the arthropods.

Insects have coexisted with freshwater and terrestrial vertebrates through most of their existence. Certain insects such as mosquitoes are parasitic to vertebrates such as humans in that the tissues and/or blood of the humans are food to insects such as mosquitoes. This close association between mosquitoes and humans has made mosquitoes nuisances which may decrease property values, affect residents and lower the overall economic potential of communities.

Additionally, insects parasitic to vertebrates may transmit diseases. For example, ticks may transmit Lyme disease, Heartwater, Congo-Crimean haemorrhagic fever and Erlichiosis; fleas may transmit Typhus and Plague; lice may transmit Trench fever; Mosquitoes may transmit malaria, Filariasis, various arbovirusses such as Yellow Fever, Dengue, Dengue Hemorrhagic Fever, Western Equine encephalomyelitis, Saint Lewis encephalitis and West-Nile encephalitis.

To eliminate or reduce the nuisance effect or disease transmission, the insect must be controlled. Three categories of “control” methods can be identified. In particular, they are chemical control, control with insect growth regulators and bacteriological control. All these methods involve aerial or surface application and have the disadvantage of non-specificity, environmental concerns, the necessity for repeated application and costs, and the danger of resistance development. In relation to non-specificity, there are negative effects on non target insects such as humans and negative effects on populations of harmless or beneficial insects.

An improvement to the control methods described above is the use of odors to attract the insect to a trap. An example is the use of moth sex pheromones to disrupt the mating through “attract and kill”, “false trail following”, or confusing males through saturating the air with pheromones. The communication channel of moths is specific and the specific blend used by certain moth species may be readily determined with Gas Chromatography coupled with Electro-Antenno Grams (EAG). In contrast, the odors involved in non-pheromone communication are more difficult to identify because EAG responses to any odor give considerably lower overall depolarization, and electrophysical responses do necessarily reflect behavioral activity.

Current odor baited insect traps for mosquitoes use carbon dioxide, which is a major component of human breath, as an attractant. Carbon dioxide is considered a primary activator and attractant for parasitic arthropods to vertebrate hosts. Currently, only those traps which utilize carbon dioxide are considered effective. Carbon dioxide is used in the form of dry ice or from pressurized cylinders. However, the use of carbon dioxide is relatively expensive and difficult to work with, and generally considered unsuitable for large-scale use in personal protection or mass trapping techniques. Additionally, carbon dioxide tends to indiscriminately attract all mosquito species. In this regard, trap catches with carbon dioxide are not very informative about the biting ‘preference’ of a species which is an important factor in a species vectorial capacity. In fact, carbon dioxide baited traps may disproportionately catch mosquitoes that are less anthropophilic and/or ornithophilic. As such, mosquito abundance and infection rates obtained from these traps may not accurately reflect actual abundance, infection rates and the risk for human epidemics.

There has been numerous studies and effort to identify odors used by arthropods that are parasitic to humans, but have not been successfully applied to traps for several reasons. First, humans have too many skin compounds. For example, glass beads which were rubbed on a human hand contained over 300 compounds. It is impossible to test all of these in the required concentration ranges and include testing for possible interactions (such as synergism) between these compounds. Second, it is important to use the right blend ratio of the compounds to form an attractive blend. Third, species differ in the precise components that they use to find their hosts, meaning that a compound or blend that is attractive to one species may not be attractive to another species even if the species prefer the same host species.

Accordingly, there is a need in the art for an improved method of producing an odor that resembles an odor of a host and may modify a behavior of an arthropod parasitic to the host.

### BRIEF SUMMARY OF THE INVENTION

In an embodiment of the present invention, a method is provided to produce a vertebrate host mimic for modifying a behavior of arthropods which are parasitic to a vertebrate host according to the steps of providing a lipid based media, providing microorganisms, combining the lipid based media and the microorganisms and collecting a modified lipid based media. The microorganisms which are associated with a skin of the host vertebrate are operative to excrete sub-products which modify the media upon combination with the lipid based media to produce modified lipid based media.

The lipid based media may contain a type of lipid found on a skin of the vertebrate host. The lipid based media may contain a type of lipid found in gland secretions of the vertebrate host. The lipid based media may contain a type of lipid found in a sebaceous gland of the vertebrate host. The type of lipid may be selected from the group consisting of glycerides, sterols, sterol esters, sterol phosphates, sterol precursors, wax, wax esters, wax alcohols, wax aldehydes and combinations thereof.

The type of lipid may be a glyceride having the formula:

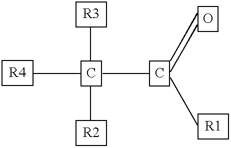

wherein R1 is selected from the group consisting of Hydroxy, Alkyloxy, Amino, Alkylamino, Diakylamino, Arylamino, Diaryloxy, Halogen and Cyano; and wherein R2, R3 and R4 is selected from the group consisting of Hydrogen, Alkyl and Aryl. The various permutations of this formula that result from hydrolysis, oxidation and esterification of especially unsaturated structures is within the scope of the present invention.

The lipid based media may further contain a type of wax having the formula:

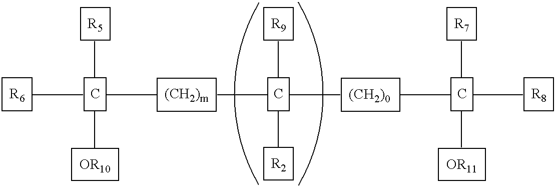

wherein R₅ to R₁₂ is selected from the group consisting of Hydrogen, Alkyl and Aryl; wherein m and o are positive integers; and wherein d is at least zero.

The lipid based media may further contain a type of sterol selected from the group consisting of sterols, sterol esters, sterol phosphates, sterol precursors and combinations thereof.

The type of lipid may be a hydrolyzed lipid. The hydrolyzed lipid may be selected from the group consisting of C10-C40 fatty acids, fatty alcohols, hydroxyacids and combinations thereof.

A synergistic component selected from the group consisting of lactic acid, fatty acids, ammonia, detones, octenol and combinations thereof may be further inserted into the modified lipid based media.

The host skin associated microorganisms may be generally distributed over the skin of the vertebrate host. The host skin associated microorganisms may be resident or transient to the skin of the vertebrate host. The host skin associated microorganisms may be capable of producing proteases, lipases, or cellulaeses. The host skin associated microorganisms may be capable of producing enzymes that hydrolyze lipids. The host skin associated microorganisms may be capable of producing enzymes that produce fatty acids. The host skin associated microorganisms may be capable of producing enzymes that produce fatty alcohols. The host skin associated microorganisms may be capable of producing enzymes that produce fatty aldehydes. The host skin associated microorganisms may be capable of producing enzymes that produce hydroxyacids.

After the microorganisms and lipid based media are combined, the microorganisms may be sterilized from the combination thereof.

In another embodiment of the present invention, a vertebrate host mimic may be produced according the method of providing lipid based media, providing microorganisms as discussed above, combining the lipid based media and the microorganisms and collecting a modified lipid based media.

In another embodiment of the present invention, a trap to ensnare arthropods which are parasitic to vertebrate hosts is provided which includes an arthropod ensnaring device and a vertebrate host mimic adjacent to the arthropod ensnaring device. More specifically, the vertebrate host mimic may be enclosed within the arthropod ensnaring device.

The vertebrate host mimic may be produced according to the steps of providing a lipid based media, providing microorganisms, combining the lipid based media and the microorganisms and collecting a modified lipid based media. The microorganisms may be associated with a skin of the host vertebrate and operative to excrete sub-products which modify the lipid based media upon combination with the lipid based media to produce modified lipid based media.

In another embodiment of the present invention, a method of producing a vertebrate host mimic for modifying a behavior of arthropods which are parasitic to a vertebrate host is provided which includes the steps of providing a lipid based media, providing enzymes, combining the lipid based media and the enzymes, and collecting a modified lipid based media. The enzymes may be of a type excreted by microorganisms associated with a skin of the host vertebrate, and the excreted enzymes may be operative to modify the lipid based media upon combination with the lipid based media to produce the modified lipid based media.

The lipid based media may contain a type of lipid found on a skin of the vertebrate host. The lipid based media may contain a type of lipid found in gland secretions of the vertebrate host. The lipid based media may contain a type of lipid found in a sebaceous gland of the vertebrate host.

The type of lipid may be selected from the group consisting of glycerides, sterols, sterol esters, sterol phosphates, sterol precursors, wax, wax esters, wax alcohols, wax aldehydes and combinations thereof. The glyceride may have the formula:

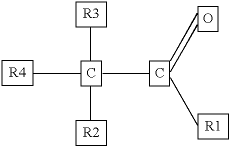

wherein R1 is selected from the group consisting of Hydroxy, Alkyloxy, Amino, Alkylamino, Diakylamino, Arylamino, Diaryloxy, Halogen and Cyano; and wherein R2, R3 and R4 is selected from the group consisting of Hydrogen, Alkyl and Aryl. The various permutations of this formula that result from hydrolysis, oxidation and esterification of especially unsaturated structures is within the scope of the present invention.

The lipid based media may further contain a type of wax having the formula:

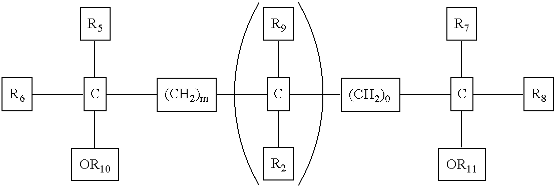

wherein R₅ to R₁₂ is selected from the group consisting of Hydrogen, Alkyl and Aryl; wherein m and o are positive integers; and wherein d is at least zero.

The lipid based media may further contain a type of sterol selected from the group consisting of sterols, sterol esters, sterol phosphates, sterol precursors and combinations thereof.

The type of lipid may be a hydrolyzed lipid. The hydrolyzed lipid may be selected from the group consisting of C10-C40 fatty acids, fatty alcohols, hydroxyacids and combinations thereof.

The method of producing the vertebrate host mimic may further include the step of inserting a synergistic component with the modified lipid based media selected from the group consisting of lactic acid, fatty acids, ammonia, detones, octenol and combinations thereof.

The host skin associated microorganisms may be a type of microorganism generally distributed over the skin of the vertebrate host. The host skin associated microorganisms may be resident or transient to the skin of the vertebrate host.

The host skin associated microorganisms may be capable of producing proteases, lipases, or cellulaeses. The host skin associated microorganisms may be capable of producing enzymes that hydrolyze lipids. The host skin associated microorganisms may be capable of producing enzymes that produce fatty acids. The host skin associated microorganisms may be capable of producing enzymes that produce fatty alcohols. The host skin associated microorganisms may be capable of producing enzymes that produce fatty aldehydes. The host skin associated microorganisms may be capable of producing enzymes that produce hydroxyacids.

The method of producing the vertebrate host mimic may further include the step of sterilizing the microorganisms.

In another embodiment of the present invention, a vertebrate host mimic may be produced according the steps of providing a lipid based media, providing enzymes, combining the lipid based media and the enzymes, and collecting the modified lipid based media. The enzymes may be of a type excreted by microorganisms associated with a skin of the host vertebrate and the excreted enzymes microorganism may be operative to modify the lipid based media upon combination with the lipid based media to produce modified lipid based media.

### BRIEF DESCRIPTION OF THE DRAWINGS

These, as well as other features of the present invention, will become more apparent upon reference to the drawings wherein:

FIG. 1 is flow chart of a method of producing a vertebrate host mimic, according to an aspect of the present invention;

FIG. 2 is an illustration of an ecosystem including a host and an arthropod parasitic to the host to show a general location where types of microorganisms and lipid based media may be found and provided in steps 12 and 14 of FIG. 1;

FIG. 3 is a petridish with microorganisms in combination with lipid based media which illustrates a combining step 16 of FIG. 1;

FIG. 4 is a cut out view of a skin of a human host to show a specific location where the types of microorganisms and lipid based media may be found and provided steps 12 and 14 of FIG. 1;

FIG. 5 is a dome trap with the vertebrate host mimic produced according to the method steps shown in FIG. 1 therein, according to another aspect of the present invention;

FIG. 6 is a cylinder trap with the vertebrate host mimic produced according to the method steps shown in FIG. 1 therein, according to another aspect of the present invention;

FIG. 7 is a bucket trap with the vertebrate host mimic produced according to the method steps shown in FIG. 1 therein, according to another aspect of the present invention;

FIG. 8 is a box omni trap with the vertebrate host mimic produced according to the method steps shown in FIG. 1 therein, according to another aspect of the present invention;

FIG. 9 is a vane trap with the vertebrate host mimic produced according to the method steps shown in FIG. 1 therein, according to another aspect of the present invention;

FIG. 10 is box trap with the vertebrate host mimic produced according to the method steps shown in FIG. 1 therein, according to another aspect of the present invention;

FIG. 11 is a perspective view of a wind tunnel for testing the behavioral modification effect on parasitic arthropods of the vertebrate host mimic as produced according to the method steps of the present invention shown in FIG. 1;

FIG. 12 is a graph of the amount of time that parasitic arthropods took to activate once presented with an odor of the vertebrate host mimic which is a result of a test performed with the wind tunnel of FIG. 11; and

FIG. 13 is a graph illustrating the percentage of parasitic arthropods that found a source of an odor which is a result of another test performed with the wind tunnel of FIG. 11.

### DETAILED DESCRIPTION OF THE INVENTION

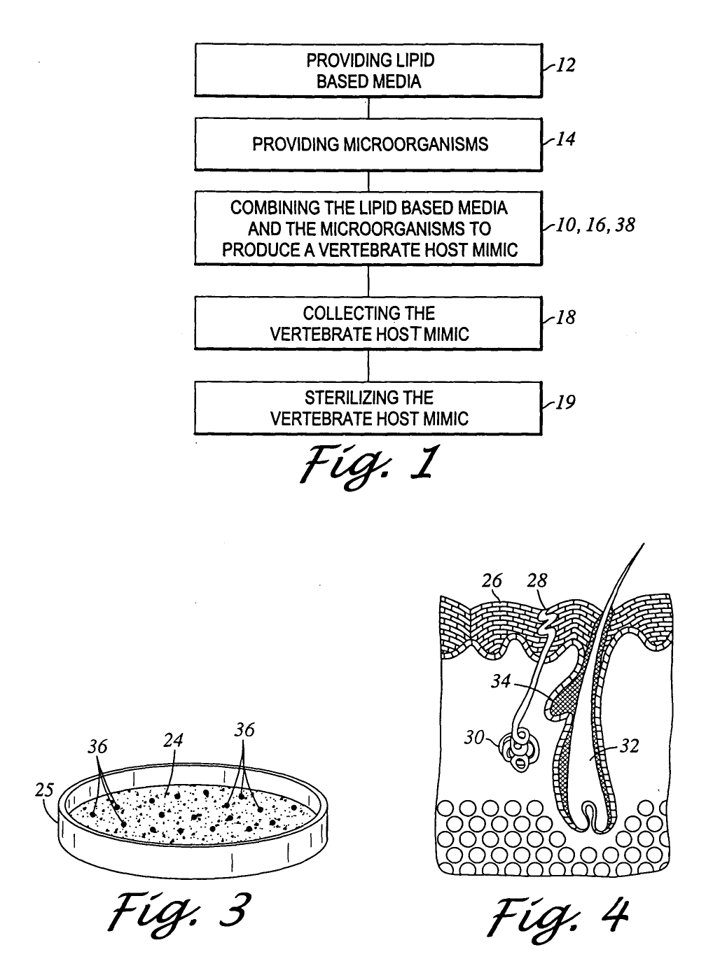

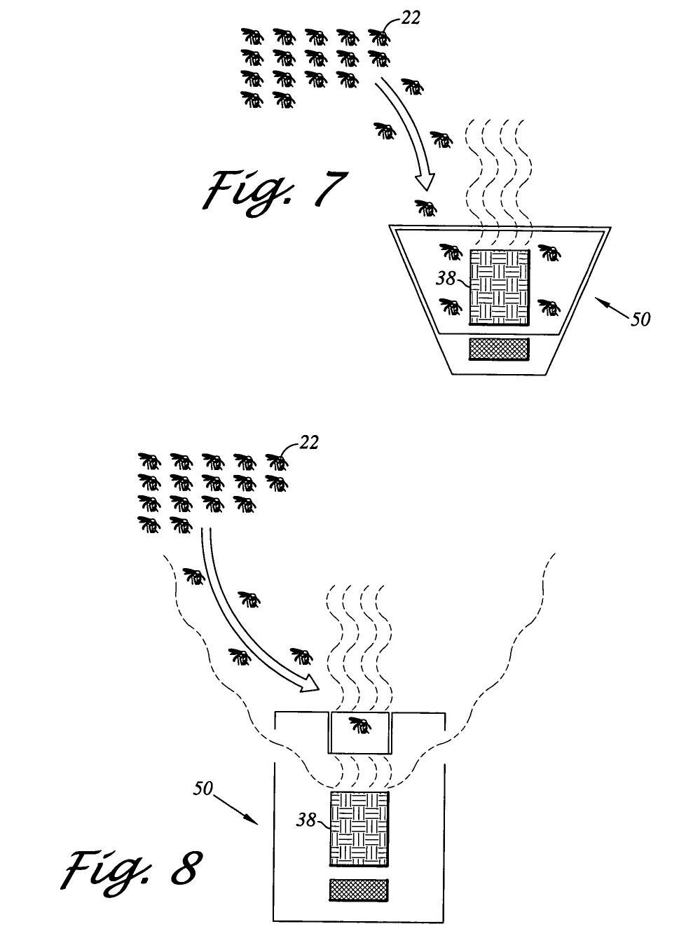

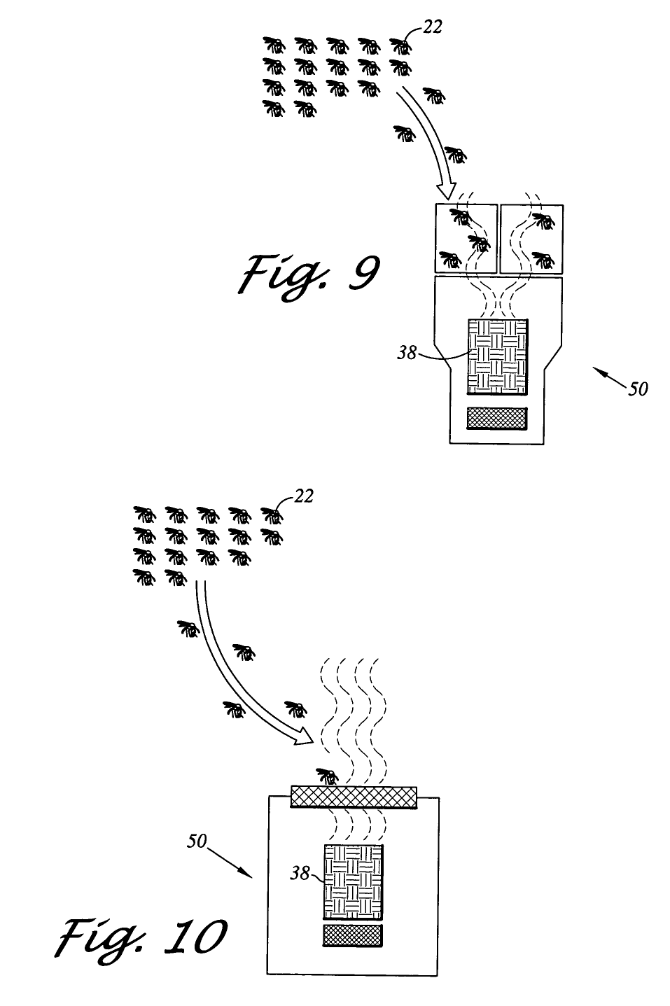

FIGS. 1-13 are for the purpose of illustrating the preferred embodiments according to aspects of the present invention, and not for the purpose of limiting the same.

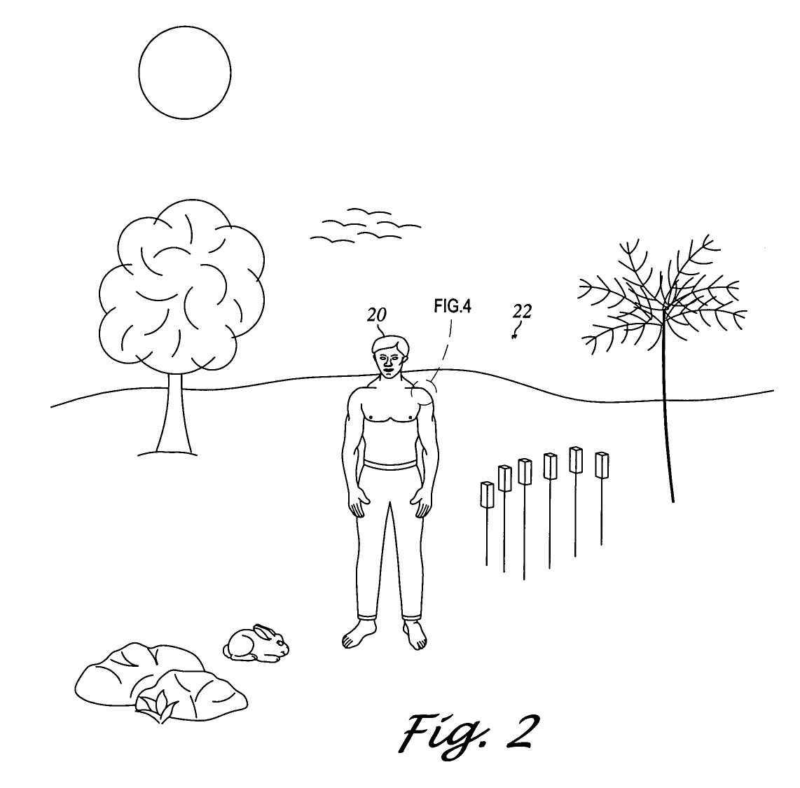

Referring now to FIG. 1 which illustrates a flow chart of a method of producing a vertebrate host mimic, in an embodiment of the present invention, there is provided a method of producing a vertebrate host mimic 10. The method includes steps 12 of providing a lipid based media, step 14 of providing microorganisms, step 16 of combining the microorganisms and the lipid based media to produce modified lipid based media 38, step 18 of collecting the modified lipid based media 38, and step 19 of sterilizing the microorganisms. The modified lipid based media 38 is the vertebrate host mimic 10. Referring now to FIG. 2 which illustrates an ecosystem including a host 20 and an arthropod 22 parasitic to the host 20, the vertebrate host mimic 10 resembles the odor of the host 20 wherein the vertebrate host mimic 10 modifies a behavior of arthropods 22. Referring now to FIG. 3 which depicts a petridish 25 with microorganisms 36 provided in step 12 and lipid based media 24 provided in step 14, the microorganisms 36 and lipid based media 24 are combined as shown in step 16 of FIG. 1.

As used herein, behavior refers to any behavior that is associated with the life cycle of the parasitic arthropod 22. By way of example and not limitation, behavior may be activation, attraction, attraction repellency, attraction orientation, attraction landing, attraction probing, attraction imbibing, and attraction oviposition. Modifies or modification refer to an influence of a substance on the behavior of the parasitic arthropod 22.

A description of the types of host 20 for which a vertebrate host mimic 10 may be formulated will be given. The host 20 may be from the subphylum vertebrata. The host 20 may be from the class reptilia, aves (i.e., birds) or mammalia. For example, the host 20 may be a human within the genus-species Homo sapien, as shown in FIG. 2, or a rat.

The lipid based media 24 (see FIG. 3) may be found on the host 20 or may be of a type of lipid based media found on the host 20. The lipid based media 24 may be found on a variety of areas on the host 20 such as the excretory organs of the host 20. Generally, the host 20 may have a plurality of excretory organs such as lungs, skin and urinary system. In particular, the lipid based media 24 may be found specifically on the host skin, as shown more clearly in FIG. 4. FIG. 4 is a cut out view of a skin of a human host 20 to show a specific location where the types of microorganisms 36 and lipid based media 24 may be found and provided in steps 12 and 14 of FIG. 1. FIG. 4 illustrates a thickened epidermis 26, a sweat pore 28, a sweat gland 30, a hair follicle 32, and a sebaceous gland 34.

In relation to human hosts 20, the lipid based media 24 may be found on the human skin or may be a type of lipid based media 24 found on the human skin. For example, a type of lipid based media 24 found on the human skin is lipids. Generally, lipids are a class of substances that are insoluble in water (and other polar solvents), but are soluble in nonpolar substances (like ether or chloroform). Three major groups of lipids are (1) triglycerides which include fats, oils and waxes, (2) phospholipids, and (3) steroids.

The human skin has sebaceous glands 34. The sebaceous gland 34 is any of the various glands in the corium of the skin that open into a hair follicle 32 that produce and secrete sebum. The lipid based media 24 may include lipids produced from the sebaceous glands 34.

Examples of lipids produced from sebaceous glands 34 of humans may be sebum which contains relatively high levels of triglycerides. According to the molecular weight, the triglycerides of human sebum should correspond to an average C54-55. The amount of sebum produced is about 1-2 g/day. When the sebum reaches the skin, the triglycerides contained within the sebum are hydrolyzed by lipases which accounts for the relatively high levels of fatty acids on the human skin. Examples of fatty acids are iso, anteiso and other mono and dimethyl branched fatty acids. Additionally, in this regard, the human skin contains relatively high levels of free fatty acids such as nC16 (palmitic, 25%), Mysteric (nC14), stearic (nC18) and oleic (nC18:1delta9) and linoleic (nC18:2delta 9, 12). Furthermore, the lipid based media may be a fatty acid having the structure of a long chain hydrocarbon chain wherein the chain length ranges from 4 to 30 carbons, preferably, 12-24 carbons, and the chain is typically linear. The lipid based media 24 may be a fatty acid having the structure of a carboxylic acid group.

The human skin has relatively high levels of wax monoesters which range from C26-42 and are predominately C34-36. The human skin secretes precursors to cholesterol such as unsaturated C30 hydrocarbon commonly referred to as squalene. In this regard, the lipid based media 24 may be wax monoesters and precursors to cholesterol.

The types of lipid based media 24, as discussed above, may be types of lipids found on or in the host skin such as triglycerides for human hosts 20. Additionally, another type of lipid based media 24 may be a precursor of a lipid such as squalene. Moreover, another type of lipid based media 24 may be a product of a lipid such as free fatty acid which is produced when sebum reaches the skin surface of the human host 20. In other words, the lipid based media 24 is associated with lipids found on the host skin, e.g., human skin.

The following is a discussion of the forms of lipid based media 24 in which the types of lipid based media 24 may be provided. In particular, the lipid based media 24 may comprise actual host related products (i.e., non-artificial compositions) such as skin rubbings, sweat and/or urine which are relatively difficult to obtain in any significant quantities. Additionally and alternatively, the lipid based media 24 may contain vertebrate host skin related products such as blood, urine, cheese, milk, lard, egg-white, hair and/or combinations of these products. Furthermore and in the alternative, the lipid based media 24 may comprise artificial compositions which may be relatively easy to obtain or produce.

The following are examples of the types of lipids. By way of example and not limitation, the lipid based media 24 may be selected from the group consisting of triglycerides, sterols, sterol precursors, squalene, wax diesters, wax monoesters, wax trimesters, stero esters, and combinations thereof. In relation to triglycerides, the same may be in a pure form or a natural raw product which contains triglycerides such as lard. Additionally, the lipid based media 24 may be pure waxes. Alternatively, the lipid based media 24 may be a raw natural product which contains relatively high levels of waxes such jojoba bean oils. Furthermore, the lipid based media 24 may be pure sterols. Alternatively, the lipid based media 24 may be cell membranes, lard or nervous tissue. In sum, the lipid based media 24 may be in their purified form, raw natural product form or a combination of the purified and raw natural product form.

The artificial compositions may be an aqueous or non-aqueous solution. The artificial compositions may contain organic, inorganic or chemical matter. The artificial compositions may comprise sources of various chemically defined and/or chemically undefined and complex compositions. Examples of chemically defined compositions are macronutrients, micronutrient and growth factor. For example, the artificial compositions may be any macronutrient source of carbon, hydrogen, oxygen, nitrogen, phosphorous, sulfur, potassium, magnesium, sodium or calcium. The artificial compositions may be any micronutrient source of chromium, cobalt, copper, manganese, molybdenum, nickel, selenium, tungsten, vanadium, or iron. The artificial compositions may be any growth factor source of organic compounds such as vitamins, amino acids, purines or pyrimidines.

Examples of chemically undefined and complex compositions are digest of casein (i.e., milk protein), beef, soybeans, yeast cells or any of a number of other nutritious and highly nutritious compositions.

Generally, the lipid based media 24 discussed above are not attractive to parasitic arthropods. In particular, the lipid based media 24 such as triglycerides for human hosts 20 are not attractive to arthropods 22 which are parasitic to human hosts 20.

The lipid based media 24 is part of the cellular respiration of the microorganisms 36. In particular, the lipid based media 24 is nutrients for the microorganisms 36. The microorganisms 36 may be survivable on the lipid based media 24 in that the microorganisms 36 may consume some of the lipid based media 24 and excrete sub-products which modify the lipid based media 24. It has been found that suitable microorganisms 36 are found where the lipid based media 24 may be found (e.g., on the human skin), or may be a type of microorganisms 36 found where the lipid based media 24 may be found. Suitable microorganisms are effective in producing modified lipid based media when combined with the lipid based media.

In particular, the microflora (e.g., bacteria and fungi that inhabit the human skin) of the human host skin may be generally divided into three groups, namely, gram-positive cocci, diphtheroid-like organisms and fungi. Certain skin microflora may be generally found over the entire skin surface, and other certain skin microflora may be found in high density over certain specific portions of the skin. For example, gram-positive cocci can be recovered from nearly all body sites such as the Staphylococcus epidermis which is one of the most predominant. Propioni spp. may be found over the entire skin. In relation to diphtheroid-like organisms, three families of diphtheroid are commonly found on the skin. In particular, Brevibacterium spp. are found mainly in the toe webs, Corynebacterium spp. are found over the entire skin. Various micrococcal species are found in high density in the armpit. Propionibaterium spp. are found over the entire skin. Preferably, the microorganism 36 may be found generally over the human skin such as gram-positive cocci and Propioni spp.

In relation to human hosts 20, the microorganisms 36 may be coagulase negative cooci, P. acnes and yeast-like fungi of the genus Pitysporum. These microorganisms 36 may be found in the sebaceous glands 34 associated with the hair follicles 32 which are found generally over the skin of the human host 20.

As stated above, effective microorganisms 36 may survive on the excretory organ of the host 20, e.g., human skin and may be found generally over the human skin. In this regard, the microorganism 36 may be capable of surviving residently or transiently within the range of conditions found on the skin of the host 20. For example, the local conditions of the skin of the host 20 may vary in relation to humidity, acidity (i.e., pH), temperature, and density of the various skin glands. With respect to human hosts 20, the human skin is relatively acidic in the range of 4-6 pH. As such, the microorganism 36 may be expected to survive in an environment having an acidity of 4-6 pH.

The following are examples of microorganisms 36 that are survivable in environments similar to the environment of the skin of the human host 20. The microorganisms 36 may be a prokaryote (i.e, a cell that does not have a nucleus or other membrane-bound organelles) and/or a eukaryote (i.e., a cell that contains an organized nucleus and other membrane-bound organelles). The microorganism may be a microorganism within the protista kingdom, eubacteria kingdom and fungi kingdom. The eubacteria may be a bacilli which is rod-shaped, cocci which is round shaped, and spirilla which is spiral shape. The eubacteria may remain attached such as the diplococci occurring in pairs, the streptococci occurring in chains and the staphylococci occurring in clusters.

The above described microorganisms 36 are capable consuming the lipid based media 24 and capable of modifying the lipid based media 24 through sub-products excreted by the microorganisms 36. Examples of the secreted sub-products are proteases, lipases, and/or cellulaeses which are enzymes that break down the lipid based media 24. In particular, propionibateria produce a variety of extracellular enzymes that include lipases, phosphatases, hyaluronate lyase, protease and others.

The enzymes produced by the microorganisms 36 found on the excretory organ of the host 20 modify the lipid based media 24 to transform the lipid based media 24 into the vertebrate host mimic 10. In this regard, the lipid based media 24 and the microorganisms 36 may be combined to produce the vertebrate host mimic 10. Alternatively, the lipid based media 24 and the types of enzymes produced by the microorganisms 36 may be combined to produce the vertebrate host mimic 10.

The following are more examples of the types of microorganisms 36. The microorganism 36 may be associated with the host 20. For example, microorganism 36 associated with the host 20 are microorganisms 36 such as Staphylococcus, Corynebacterium, Acetinobacter, Pityrosporum, Propionibacterium, Streptococcus, Lactobacillus, Fusobacterium, Velillonella, Actinomycetes, Neisseria, Bacteroides, Escherichia, Proteus, Bacilli, Klebsiella, Clostridium, Eubacterium, Ruminococcus, Brevibacterium, Mycobacterium, Actinomyces, Dermatophilus and combinations thereof.

The microorganism 36 may be a prokaryotic microorganism such as a bacteria. For example, the microorganism 36 may be selected from the group of bacteria consisting of Gram-Negative Bacteria, gram-positive bacteria, Archaea, Filamentous actinomycetes and combinations thereof.

Additionally, the microorganism 36 may be selected from the group of bacteria consisting of Spirochetes, Aerobic/Micoraerophilic, Motile, Helical/Vibrioid Gram-negative Bacteria, Nonemotile Gram-Negative Curved Bacteria, Gram-negative Aerobic Rods, Gram-negative Aerobic Cocci, Facultative Anaerobic Gram-Negative Rods, Enterobacteriaceae, Anaerobic Gram-Negative Straight, Anaerobic Gram-Negative Curved, Anaerobic Gram-Negative Helical Rods, Dissimilatory Sulfate Reducing Bacteria, Dissimilatory Sulfur Reducing Bacteria, Anaerobic Gram-Negative Cocci, Ricketsias, Chlamydias, Mycoplasmas and combinations thereof.

The microorganism 36 may be selected from the group of bacteria consisting of Gram-positive cocci, Endospore-Forming Gram Positive Rods, Endospore-Forming Gram Positive Cocci, Regular Non-Spore Forming Gram-Positive Rods, Irregular nonsporing Gram Positive Rods, Mycobacteria, Nocardioforms, Anoxygenic Phototrophic Bacteria, Oxygenic Photosynthetic Bacteria, Aerobic Chemolithotrophic bacteria, Budding and/or Appendaged Bacteria, Sheated Bacteria, Nonphotosynthetic Nonfruiting Gliding Bacteria, Fruiting Bacteria and combinations thereof.

The microorganism 36 may be selected from the group of bacteria consisting of Methanogenic Archaeobacteria, Archaeobacterial Sulfate Reducers, Extremely Halophilic Archaeobacteria, Nocardioform Actinomycetes, Actinomycetes with Mutipcellular Sporangia, Actinoplanetes, Streptomycetes, Maduromycetes and combinations thereof.

The microorganism 36 may be a eukaryotic microorganism such as a fungi. For example, the microorganism 36 may be selected from the group of fungi consisting of Myxomycota, Mastigomycotina, Zygomycotina, Ascomycotina, Basidiomycotina, Deuteromycotina, Oomycetes. Additionally, the microorganism 36 may be selected from the group of algae consisting of Chlorophyta, Euglenophyta, Chrysophyta, Phaeophyta, Pyrrophyta, Rhodophyta. Alternatively, the microorganism 36 may be an algae.

As mentioned above in combining step 16, after the lipid based media 24 and microorganisms 36 are provided, they are combined 16. For example, the lipid based media 24 and microorganisms 36 may be combined as shown in FIG. 4 in a petridish 25. In other words, they are allowed to form a biological culture. Preferably, the period of combination (i.e., incubation) is about three days. The environment in which they are combined is controlled. By way of example and not limitation, the environment is controlled with respect to pH, humidity and temperature.

The combination described above of the lipid based media 24 and microorganisms 36 or enzymes may be essentially free of carbon dioxide in that the commercialized versions thereof such as carbon dioxide in the form of dry ice or pressurized cylinders are not introduced in the fumes created by the combination. Additionally, the combination of lipid based media 24 and microorganisms 36 or enzymes may be essentially free of lactic acid in that the purified forms or commercialized forms thereof are not introduced in the combination.

The method of producing the vertebrate host mimic 10 as flow charted in FIG. 1 may further include step 19 of sterilizing the microorganisms 36 after the microorganisms 36 are combined with the lipid based media 24. The microorganisms 36 are sterilized when the microorganisms 36 are killed, removed or inactivated from the combination. In this regard, the combination is no longer a biological culture. Even though the microorganism 36 has been sterilized from the combination, the transformed media is similar to the odor profile of the host.

As mentioned above in collecting step 18, after the lipid based media 24 has been modified by the microorganisms 36 or enzymes, then the modified lipid based media 38 may be collected. In this regard, the modified lipid based media 38 may be collected before the microorganisms 36 are sterilized. Preferably, the modified lipid based media 38 is collected after the microorganisms 36 are sterilized. The reason is that living cultures of microorganisms 36 may be a health issue to those who use the vertebrate host mimic 10.

The modified lipid based media 38 may be collected into a hermetically sealed container. The container may be flexible or rigid container. The container may have a regulatable open top in that an orifice of the open top may be made larger or smaller as a function of the amount of released odor from the modified lipid based media which is desired. The container may be a timed release canister which releases a quantity of odor from the modified lipid based media 38 at regular intervals of time. The container may have a semipermeable membrane. The container may allow the modified lipid based media 38 to be emptied from the container regardless of whether the modified lipid based media 38 is in the aqueous or coagulated form. The modified lipid based media 38 may be emptied from the container either through the physical pouring out with the aid of gravity acting on the modified lipid based media 38, or through placing the modified lipid based media 38 in a pressurized container such as a spray paint can. When the modified lipid based media 38 is in the container, the air or gas within the container is preferably evacuated therefrom.

The container may be opaque to an extent that a user may not be able to identify the contents of the container. Alternatively, the container may be clear to an extent that the user may be able to identify the contents of the container. In this regard, the modified lipid based media 38 may be colored with an inert material which does not affect the ability of the same to modify the behavior of the parasitic arthropod 22.

The modified lipid based media 38 may be collected in absorbent material which retards the release of the modified lipid based media 38 such as fabric, paper, porous material, foan, absorbent polymer, super absorbent polymer.

The vertebrate host mimic 10 may comprise the modified lipid based media 38 and additional components to increase its behavior modification effect on parasitic arthropods 22. For example, the vertebrate host mimic may further contain lactic acid, fatty acids, ammonia, ketones, and octenol and carbon dioxide.

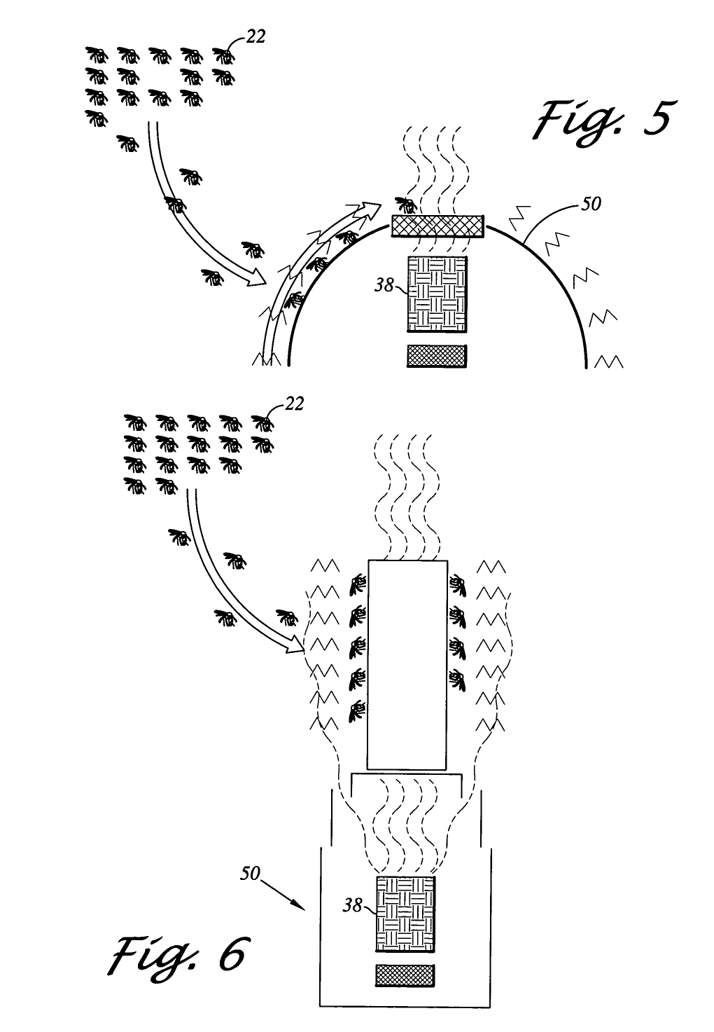

A trap 50 is a device which ensnares an arthropod 22. In an embodiment of the present invention, traps 50 may be used to capture parasitic arthropods 22. For example, the modified lipid based media 38 may be placed adjacent to a trap 50 having a construction selected from the group consisting of dome trap, cylinder trap, bucket trap, box omni trap, vane trap and box trap which are shown in FIGS. 5-10, respectively. The modified lipid based media 38 may be placed adjacent to the trap 50 with or without a gas stream (e.g., air, carbon dioxide, an airstream, or other equivalent gaseous stream) as a carrier. More specifically, by way of example and not limitation, the modified lipid based media 38 may be placed within the dome trap 50, as shown in FIG. 5.

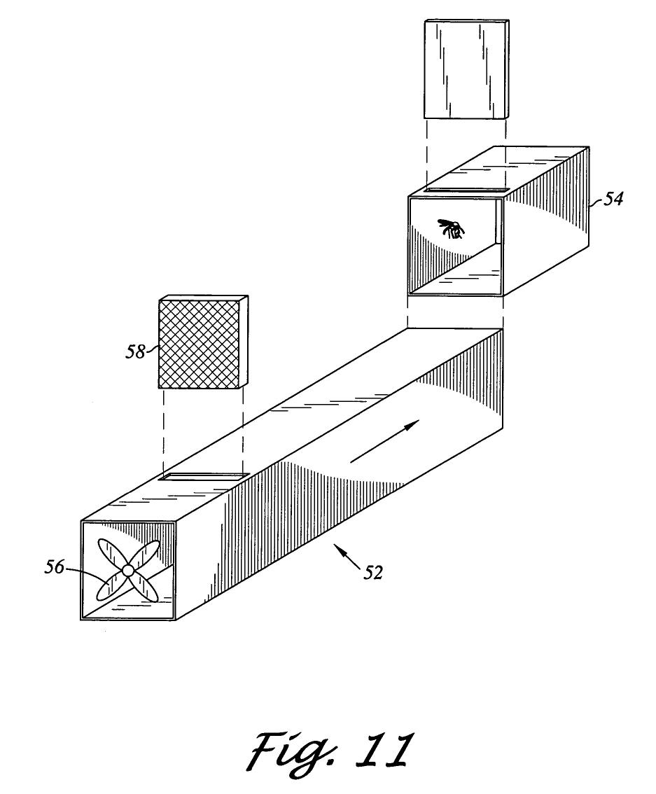

Referring now to FIG. 11, a wind tunnel 52 is shown which is used for testing the behavioral modification effect on parasitic arthropods 22 of the vertebrate host mimic 10. The wind tunnel 52 consisted of Plexiglas walls and had a dimension of 60 (breadth)×60 (height)×200 (length) cm. The air flow in the wind tunnel 52 was laminar having a flow rate of about 40 cm/s flow. The temperature was about 25 to 29 degrees Celcius. The air was filtered through an activated charcoal filter (not shown). The activated charcoal filter was placed between a fan 56 and screen 58.

Parasitic arthropods 22, specifically, 10-30 day old nulliparious female Aedes aegypti were placed downstream in the wind tunnel 52. The Aedes aegypti were reared in climate controlled chambers at 80% RH and L:D 12:12. Adult Aedes aegypti were kept in 30 cm by 30 cm by 30 cm gauze cages 54 and provided with 6% sucrose in water. Larvae were reared in trays at an approximate density of 1 per 1 cm² water surface area. The Aedes aegypti were reared on rabbit pellets and fish food similar to ones sold under the trademark TETRAMIN.

Four treatments were used, namely unmodified lipid based media (i.e., control), modified lipid based media which is relatively rich in lipid based media (i.e., rich), modified lipid based media which is relatively poor in lipid based media (i.e., poor) and host hand. The treatments were impregnated into polyester screens 58 by either rubbing them thoroughly with human hands in relation to human hosts 20 or by placing the screens 58 in aqueous solutions of the control, rich and poor for about 1 minute and air drying the screens 58.

Two hours prior to testing, the Aedes aegypti were transferred to release cages 54. At the time of the test, the treated screens 58 were placed one at a time upstream into the wind tunnel 52, and the release cages 54 were placed downstream in the wind tunnel 52 for two minutes to climatize. Thereafter, the Aedes aegypti were released into the air flow.

The amount of time in seconds for the Aedes aegypti to activate (i.e., to go from quiescent to take off in flight) after the screens 58 and cages 54 were placed in the wind tunnel were observed. The average time for activation is shown in FIG. 12. In particular, the average time for activation was about 19 seconds, 20 seconds, and 17 seconds for 80% or more of the Aedes aegypti after the Aedes aegypti were released from the cages and presented with screens treated with rich media, poor media and hand, respectively. In this regard, the activation time is indistinguishable between the different tests. As such, this test indicates that Aedes aegypti females cannot distinguish between the natural (i.e., hand) and the modified lipid based media. In contrast, the average time for activation was about 120 seconds for about 25% of the Aedes aegypti after the Aedes aegypti were released from the cages and presented with screens treated with control.

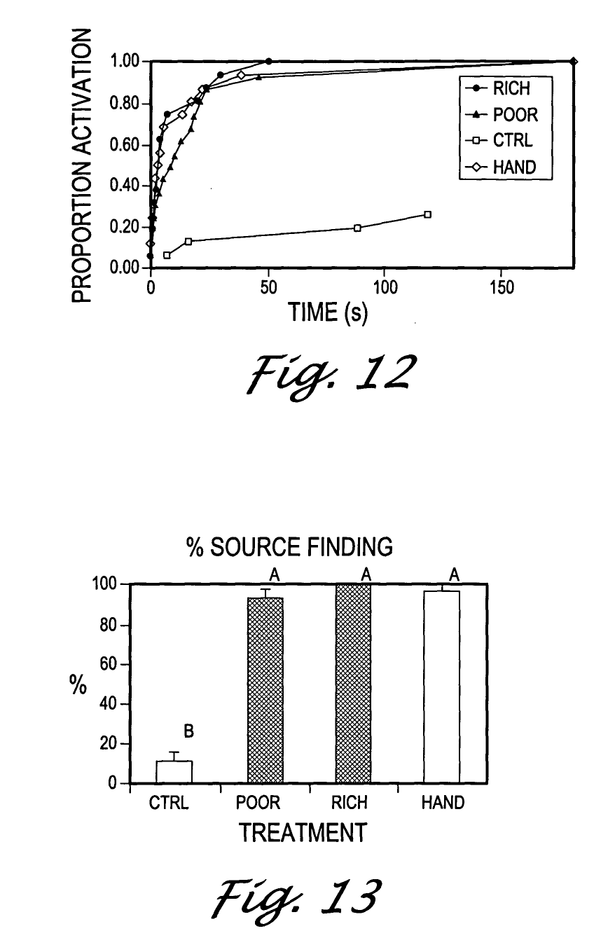

The number of Aedes aegypti finding the source (i.e., treated screens) after the screens 58 and cages 54 were placed in the wind tunnel 52 were observed. The percentage of Aedes aegypti finding the source is shown in FIG. 13. The error bars represent the standard deviation. About the same percentage of Aedes aegypti found the source in relation to poor media (i.e., 93%), rich media (i.e., 100%) and hand (i.e., 96.4%). In contrast, only 11% of the Aedes aegypti presented with the control found the source. As such, this test indicates that Aedes aegypti females do not distinguish between the natural (i.e., hand) and the modified lipid based media 38.

In summary, evolution has made certain arthropods 22 parasitic to particular hosts 20. Certain parasitic arthropods 22 are parasitic to the whole skin of the host 20. In this regard, the lipid based media 24 is based on lipids or derivations thereof which are found generally over the whole skin of the host 20. It is noted that lipid based media 24 prior to being combined with the microorganisms 36 do not act to modify the behavior of the parasitic arthropods 22. After the lipid based media 24 is selected and provided, the microorganisms 36 are combined with the lipid based media 24. The combination of the lipid based media 24 and the microorganism 36 may be in any form so long as the microorganisms 36 are allowed to consume at least a portion of the lipid based media 24 and sub-products (e.g., enzymes) excreted by the microorganisms 36 may break down the lipid based media 24. In this regard, the lipid based media 24 that was not capable of modifying the behavior of parasitic arthropods 22 is now transformed into a modified lipid based media 38 that is capable of modifying the behavior of parasitic arthropods 22. The modified lipid based media 38 may be collected in many different forms such as being soaked up or placed in a spray can.

Additional modifications and improvements of the present invention may also be apparent to those of ordinary skill in the art. Thus, the particular combination of parts and steps described and illustrated herein is intended to represent only one embodiment of the present invention, and is not intended to serve as limitations of alternative embodiments and methods within the spirit and scope of the present invention.

What is claimed is:

1. A method of producing a vertebrate host mimic for modifying a behavior of arthropods which are parasitic to a vertebrate host, the method comprising the steps of:
a. providing a lipid based media;
b. providing microorganisms, the microorganisms being associated with a skin of the host vertebrate and operative to excrete sub-products which modify the media upon combination with the lipid based media to produce modified lipid based media;
c. combining the lipid based media and the microorganisms; and
d. collecting the modified lipid based media.

2. The method of claim 1 wherein the lipid based media contains a type of lipid found on a skin of the vertebrate host.

3. The method of claim 2 wherein the lipid based media contains a type of lipid found in the gland secretions of the vertebrate host.

4. The method of claim 3 wherein the lipid based media contains a type of lipid found in the sebaceous gland of the vertebrate host.

5. The method of claim 1 wherein the lipid based media is a type of lipid selected from the group consisting of glycerides, sterols, sterol esters, sterol phosphates, sterol precursors, wax, wax esters, wax alcohols, wax aldehydes and combinations thereof.

6. The method of claim 1 wherein the lipid based media is a type glyceride having the formula: 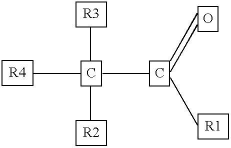
b. wherein R1 is selected from the group consisting of Hydroxy, Alkyloxy, Amino, Alkylamino, Diakylamino, Arylamino, Diaryloxy, Halogen and Cyano; and
c. wherein R2, R3 and R4 is selected from the group consisting of Hydrogen, Alkyl and Aryl.

7. The method of claim 1 wherein the lipid based media is a type of wax having the formula:
a. 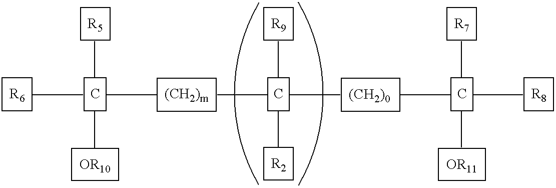
b. wherein R₅ to R₁₂ is selected from the group consisting of Hydrogen, Alkyl and Aryl;
c. wherein m and o are positive integers; and
d. wherein d is at least zero.

8. The method of claim 1 wherein the lipid based media is a hydrolyzed lipid.

9. The method of claim 8 wherein the hydrolyzed lipid is selected from the group consisting of C10-C40 fatty acids, fatty alcohols, hydroxyacids and combinations thereof.

10. The method of claim 1 wherein the host skin associated microorganisms are generally distributed over the skin of the vertebrate host.

11. The method of claim 10 wherein the host skin associated microorganisms are resident and transient to the skin of the vertebrate host.

12. The method of claim 1 wherein the host skin associated microorganisms are capable of producing proteases, lipases, or cellulaeses.

13. The method of claim 1 wherein the microorganisms are capable of producing enzymes that hydrolyze lipids.

14. The method of claim 1 wherein the microorganisms are capable of producing enzymes that produce fatty acids.

15. The method of claim 1 wherein the microorganisms are capable of producing enzymes that produce fatty alcohols.

16. The method of claim 1 wherein the microorganisms are capable of producing enzymes that produce fatty aldehydes.

17. The method of claim 1 wherein the microorganisms are capable of producing enzymes that produce hydroxyacids.

18. The method of claim 1 further comprising the step of:
e. sterilizing the microorganisms.

19. A vertebrate host mimic produced according the method of claim 1.

20. A trap to ensnare arthropods which are parasitic to vertebrate hosts, the trap comprising:
an arthropod ensnaring device; and
a vertebrate host mimic disposed adjacent the arthropod ensnaring device, the vertebrate host mimic produced according to the steps of:
a. providing a lipid based media;
b. providing microorganisms, the microorganisms being associated with a skin of the host vertebrate and operative to excrete sub-products which modify the media upon combination with the lipid based media to produce modified media;
c. combining the lipid based media and the microorganisms; and
d. collecting the modified lipid based media.

21. The trap of claim 20 wherein the vertebrate host mimic is enclosed within the arthropod ensnaring device.

22. A method of producing a vertebrate host mimic for modifying a behavior of arthropods which are parasitic to a vertebrate host, the method comprising the steps of:
a. providing a lipid based media;
b. providing enzymes, the enzymes being of a type excreted by microorganisms associated with a skin of the host vertebrate and the excreted enzymes microorganism being operative to modify the lipid based media upon combination with the lipid based media to produce modified lipid based media;
c. combining the lipid based media and the enzymes; and
d. collecting the modified lipid based media.

23. The method of claim 22 wherein the lipid based media contains a type of lipid found on a skin of the vertebrate host.

24. The method of claim 23 wherein the lipid based media contains a type of lipid found in the gland secretions of the vertebrate host.

25. The method of claim 24 wherein the lipid based media contains a type of lipid found in the sebaceous gland of the vertebrate host.

26. The method of claim 22 wherein the lipid based media is a type of lipid selected from the group consisting of glycerides, sterols, sterol esters, sterol phosphates, sterol precursors, wax, wax esters, wax alcohols, wax aldehydes and combinations thereof.

27. The method of claim 22 wherein the lipid based media is a type of glyceride having the formula:
a. 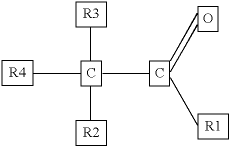
b. wherein R1 is selected from the group consisting of Hydroxy, Alkyloxy, Amino, Alkylamino, Diakylamino, Arylamino, Diaryloxy, Halogen and Cyano; and
c. wherein R2, R3 and R4 is selected from the group consisting of Hydrogen, Alkyl and Aryl.

28. The method of claim 22 wherein the lipid based media is a type of wax having the formula:
a. 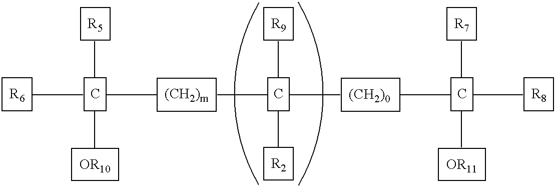
b. wherein R₅ to R₁₂ is selected from the group consisting of Hydrogen, Alkyl and Aryl;
c. wherein m and o are positive integers; and
d. wherein d is at least zero.

29. The method of claim 22 wherein the lipid based media is a type of hydrolyzed lipid.

30. The method of claim 29 wherein the hydrolyzed lipid is selected from the group consisting of C10-C40 fatty acids, fatty alcohols, hydroxyacids and combinations thereof.

31. The method of claim 22 wherein the host skin associated microorganisms are generally distributed over the skin of the vertebrate host.

32. The method of claim 31 wherein the host skin associated microorganisms are resident and transient to the skin of the vertebrate host.

33. The method of claim 22 wherein the microorganisms are capable of producing proteases, lipases, or cellulaeses.

34. The method of claim 22 wherein the microorganisms are capable of producing enzymes that hydrolyze lipids.

35. The method of claim 22 wherein the microorganisms are capable of producing enzymes that produce fatty acids.

36. The method of claim 22 wherein the microorganisms are capable of producing enzymes that produce fatty alcohols.

37. The method of claim 22 wherein the microorganisms are capable of producing enzymes that produce fatty aldehydes.

38. The method of claim 22 wherein the microorganisms are capable of producing enzymes that produce hydroxyacids.

39. The method of claim 22 further comprising the step of:
e. sterilizing the microorganisms.

40. A vertebrate host mimic produced according the method of claim 22.

41. A trap to ensnare arthropods which are parasitic to vertebrate hosts, the trap comprising:
an arthropod ensnaring device; and
a vertebrate host mimic disposed adjacent the arthropod ensnaring device, the vertebrate host mimic produced according to the steps of:
a. providing a lipid based media;
b. providing enzymes, the enzymes being of a type excreted by microorganisms associated with a skin of the host vertebrate and the excreted enzymes microorganism being operative to modify the lipid based media upon combination with the lipid based media to produce modified lipid based media;
c. combining the lipid based media and the enzymes; and
d. collecting the modified lipid based media.

42. The trap of claim 41 wherein the vertebrate host mimic is enclosed within the arthropod ensnaring device.
